(19) United States
(12) Patent Application Publication
(10) Pub. No.: US 2010/0087859 A1
(43) Pub. Date: Apr. 8, 2010

(54) FACET BUTTON ASSEMBLY AND RELATED SURGICAL METHODS

(76) Inventor: Darrell JACKSON, JR., St. Louis, MO (US)

Correspondence Address: Clements Bernard PLLC, 1901 Roxborough Road, Suite 250, Charlotte, NC, 28211, US

(21) Appl. No.: 12/572,638

(22) Filed: Oct. 2, 2009

Related U.S. Application Data
(60) Provisional application No. 61/102,594, filed on Oct. 3, 2008.

(51) Int. Cl. A61B 17/70

(52) U.S. Cl. 606/247

(57) ABSTRACT

The present invention provides a facet button assembly, including: a superior button coupled to a first member; and an inferior button coupled to a second member; wherein the first member and the second member are disposed through a bore drilled through a superior facet and an inferior facet of a facet joint of a spine of a patient and secured one of together and to the inferior button and the superior button, respectively, thereby tensioning the superior button and the inferior button together and immobilizing the facet joint of the spine of the patient. Optionally, each of the superior button and the inferior button define a bore there through, each bore configured and sized to receive one of the first member and the second member there through.

### CROSS-REFERENCE TO RELATED APPLICATION(S)

The present non-provisional patent application/patent claims the benefit of priority of U.S. Provisional Patent Application No. 61/102,594, filed on Oct. 3, 2008, and entitled “FACET BUTTON ASSEMBLY AND RELATED SURGICAL METHODS,” the contents of which are incorporated in full by reference herein.

### FIELD OF THE INVENTION

The present invention relates generally to a minimally-invasive, surgically-implantable spinal assembly. More specifically, the present invention relates to a facet button assembly that is used to immobilize a facet joint of the spine and related surgical methods.

### BACKGROUND OF THE INVENTION

In various cases, it is desirable to immobilize a facet joint of the spine (e.g. in support of a spinal fusion, etc.) by means other than conventional systems and methods. A variety of minimally-invasive, surgically-implantable systems have been developed in order to address such cases. Typically, these systems utilize a single transfacet bolt or the like that is disposed through a bore drilled through the superior and inferior facets of the facet joint. Alternatively, these systems utilize a facet cap, band, or the like that is disposed about the superior and inferior facets of the facet joint. However, not all spinal morphologies support the utilization of such a transfacet bolt, cap, or band, and the utilization of such a transfacet bolt, cap, or band can be clumsy. In such cases, the use of an alternative system is desirable. However, no such alternative systems are currently available or in existence.

### BRIEF SUMMARY OF THE INVENTION

In various exemplary embodiments, the present invention provides a facet button assembly that is used to immobilize a facet joint of the spine and related surgical methods.

In one exemplary embodiment, the present invention provides a facet button assembly, including: a superior button coupled to a lateral wire; and an inferior button coupled to a medial wire; wherein the lateral wire and the medial wire are threaded through a bore drilled through a superior facet and an inferior facet of a facet joint of a spine of a patient and secured one of together and to the inferior button and the superior button, respectively, thereby tensioning the superior button and the inferior button together and immobilizing the facet joint of the spine of the patient. Optionally, each of the superior button and the inferior button define a bore there through, each bore configured and sized to receive one of the lateral wire and the medial wire there through. Optionally, the lateral wire and the medial wire are secured together after each passes through the bore of the inferior button and the superior button, respectively.

In another exemplary embodiment, the present invention provides a facet button assembly, including: a superior button; a cannulated receptacle body hingedly connected to the superior button; an inferior button; and a plug body hingedly connected to the inferior button; wherein the cannulated receptacle body and the plug body are disposed through a bore drilled through a superior facet and an inferior facet of a facet joint of a spine of a patient and secured together, thereby tensioning the superior button and the inferior button together and immobilizing the facet joint of the spine of the patient. Preferably, the plug body is selectively disposed at least partially within the cannulated receptacle body. Optionally, an inner surface of the cannulated receptacle body and an outer surface of the plug body comprise one or more interlocking structures. Optionally, a bone engaging surface of each of the superior button and the inferior button includes one or more biting structures.

In a further exemplary embodiment, the present invention provides a facet button assembly, including: a superior button coupled to a first member; and an inferior button coupled to a second member; wherein the first member and the second member are disposed through a bore drilled through a superior facet and an inferior facet of a facet joint of a spine of a patient and secured one of together and to the inferior button and the superior button, respectively, thereby tensioning the superior button and the inferior button together and immobilizing the facet joint of the spine of the patient. Optionally, each of the superior button and the inferior button define a bore there through, each bore configured and sized to receive one of the first member and the second member there through.

### BRIEF DESCRIPTION OF THE DRAWINGS

The present invention is illustrated and described herein with reference to the various drawings, in which like reference numbers are used to denote like assembly components/method steps, as appropriate, and in which:

FIG. 1 is a schematic diagram illustrating one exemplary embodiment of the facet button assembly of the present invention, specifically a button-wire configuration; and

FIG. 2 is a schematic diagram illustrating another exemplary embodiment of the facet button assembly of the present invention, specifically a male-female button configuration.

### DETAILED DESCRIPTION OF THE INVENTION

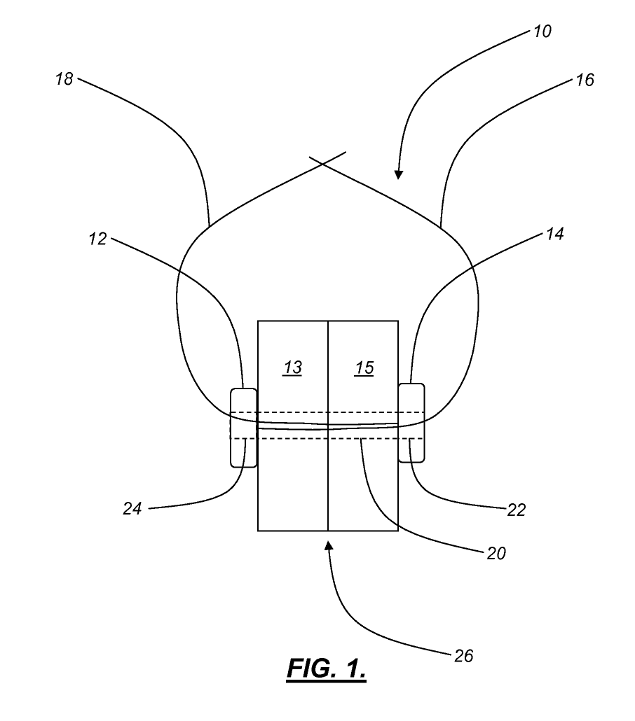

Referring now to FIG. 1, in one exemplary embodiment, the facet button assembly 10 of the present invention includes a superior button 12 and an inferior button 14. The superior button 12 is selectively disposed adjacent to the superior facet 13 of the spine of a patient. The inferior button 14 is selectively disposed adjacent to the inferior facet 15 of the spine of the patient. The superior button 12 is attached to a lateral wire 16 or other retention structure that is flexible, semi-rigid, partially rigid, or rigid and the inferior button 14 is attached to a medial wire 18 or other retention structure that is flexible, semi-rigid, partially rigid, or rigid. Prior to placement, a bore 20 is drilled through the superior facet 13 and the inferior facet 15 of the spine of the patient. The lateral wire 16 associated with the superior button 12 is threaded through the superior facet 13 and the inferior facet 15, through the bore 20. Optionally, the lateral wire 16 associated with the superior button 12 is also threaded through a bore 22 manufactured through the inferior button 14. The medial wire 18 associated with the inferior button 14 is threaded through the inferior facet 15 and the superior facet 13, through the bore 20. Optionally, the medial wire 18 associated with the inferior button 14 is also threaded through a bore 24 manufactured through the superior button 12. The facet joint 26 is immobilized by pulling the lateral wire 16 and the medial wire 18 tight and securing them, by tying them together, joining them using a fastener, etc. Optionally, the lateral wire 16 and the medial wire 18 are simply secured to the opposing button. Optionally, the lateral wire 16 and the medial wire 18 are made of Nitinol or the like, and the superior button 12 and the inferior button 14 are made of a radiolucent material, such as PEEK or the like, such that they can be medically visualized. Once placed, the superior button 12 sits snugly against the superior facet 13 and the inferior button 14 sits snugly against the inferior facet 15, the two being tensioned towards one another by one or both of the lateral wire 16 and the medial wire 18. It will be readily apparent to those of ordinary skill in the art that the lateral wire 16 and the medial wire 18 could also be replaced with bendable split brads or the like.

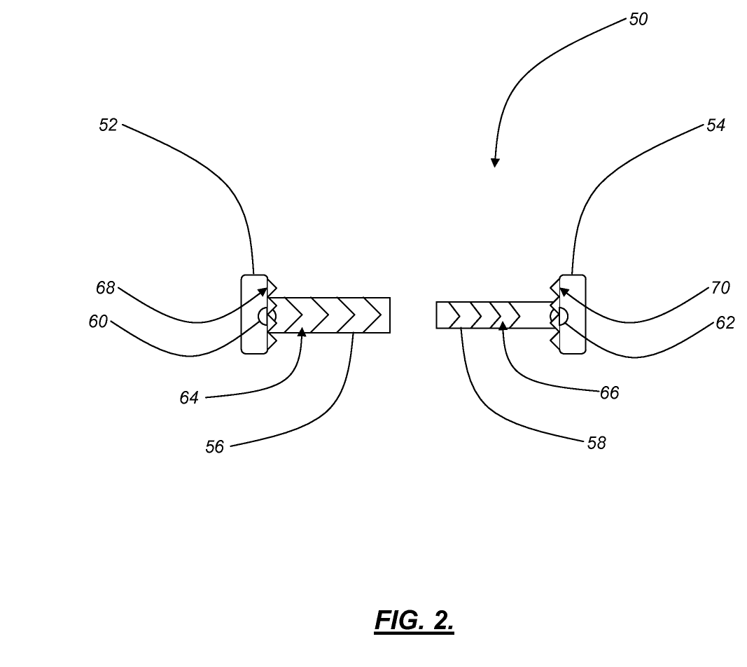

Referring now to FIG. 2, in another exemplary embodiment, the facet button assembly 50 of the present invention includes a superior button 52 and an inferior button 54. The superior button 52 is selectively disposed adjacent to the superior facet of the spine of a patient. The inferior button 54 is selectively disposed adjacent to the inferior facet of the spine of the patient. The superior button 52 is attached to a cannulated receptacle body 58 and the inferior button 54 is attached to a solid (or hollow) plug body 58. Both the receptacle body 56 and the plug body 58 are connected to their respective button 52,54 via a hinge or ball-and-socket connector 60,62, such that the receptacle body 58 and the plug body 58 can pivot in one or more directions and/or rotate relative to the respective button 52,54. The inner surface of the receptacle body 58 includes one or more raised structures 64 that are configured to engage a corresponding one or more raised structures 66 on the outer surface of the plug body 58. Prior to placement, a bore is drilled through a superior facet and an inferior facet of the spine of the patient. The receptacle body 56 associated with the superior button 52 is disposed through the superior facet and the inferior facet, through the bore. The plug body 58 associated with the inferior button 54 is disposed through the inferior facet and the superior facet, through the bore, where it engages the receptacle body 56, the raised structures 56 holding the two components securely together. The facet joint 26 is thus immobilized by pressing the plug body 58 into the receptacle body 56. Optionally, the superior button 52, the inferior button 54, the receptacle body 56, and the plug body 58 are made of a radiolucent material, such as PEEK or the like, such that they can be medically visualized. Once placed, the superior button 52 sits snugly against the superior facet and the inferior button 54 sits snugly against the inferior facet, the two being tensioned towards one another. This interface is enhanced by the pivotability of the superior button 52 and the inferior button 54. Preferably, the bone engaging surfaces of the superior button 52 and the inferior button 54 includes one or more sharp teeth or a friction surface 68,70. Thus, the receptacle body 56 and the plug body 58 act conceptually as the lateral wire 16 (FIG. 1) and the medial wire 18 (FIG. 1) described above.

Although the present invention has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the present invention, are contemplated thereby, and are intended to be covered by the following claims.

What is claimed is:

1. A facet button assembly, comprising:
a superior button coupled to a lateral wire; and
an inferior button coupled to a medial wire;
wherein the lateral wire and the medial wire are threaded through a bore drilled through a superior facet and an inferior facet of a facet joint of a spine of a patient and secured one of together and to the inferior button and the superior button, respectively, thereby tensioning the superior button and the inferior button together and immobilizing the facet joint of the spine of the patient.

2. The facet button assembly of claim 1, wherein each of the superior button and the inferior button define a bore there through, each bore configured and sized to receive one of the lateral wire and the medial wire there through.

3. The facet button assembly of claim 2, wherein the lateral wire and the medial wire are secured together after each passes through the bore of the inferior button and the superior button, respectively.

4. A facet button assembly, comprising:
a superior button;
a cannulated receptacle body hingedly connected to the superior button;
an inferior button; and
a plug body hingedly connected to the inferior button;
wherein the cannulated receptacle body and the plug body are disposed through a bore drilled through a superior facet and an inferior facet of a facet joint of a spine of a patient and secured together, thereby tensioning the superior button and the inferior button together and immobilizing the facet joint of the spine of the patient.

5. The facet button assembly of claim 4, wherein the plug body is selectively disposed at least partially within the cannulated receptacle body.

6. The facet button assembly of claim 5, wherein an inner surface of the cannulated receptacle body and an outer surface of the plug body comprise one or more interlocking structures.

7. The facet button assembly of claim 4, wherein a bone engaging surface of each of the superior button and the inferior button comprises one or more biting structures.

8. A facet button assembly, comprising:
a superior button coupled to a first member; and
an inferior button coupled to a second member;
wherein the first member and the second member are disposed through a bore drilled through a superior facet and an inferior facet of a facet joint of a spine of a patient and secured one of together and to the inferior button and the superior button, respectively, thereby tensioning the superior button and the inferior button together and immobilizing the facet joint of the spine of the patient.

9. The facet button assembly of claim 8, wherein each of the superior button and the inferior button define a bore there through, each bore configured and sized to receive one of the first member and the second member there through.
